(19) United States
(12) Patent Application Publication
(10) Pub. No.: US 2021/0346624 A1
(43) Pub. Date: Nov. 11, 2021

(54) VACUUM SHIELD

(71) Applicant: Smart RS Inc., Ottawa (CA)

(72) Inventor: Francesco FIORENZA, Ottawa (CA)

(73) Assignee: Smart RS Inc., Ottawa (CA)

(21) Appl. No.: 17/313,802

(22) Filed: May 6, 2021

(30) Foreign Application Priority Data
May 6, 2020 (CA) 3080573

(51) Int. Cl. A61M 16/00

(52) U.S. Cl. CPC A61M 16/009; A61M 16/06

(57) ABSTRACT

A vacuum shield for use on an oxygen therapy mask comprising a semi-rigid shield having outer wall regions for covering the oxygen therapy mask and exhaust ports of the oxygen therapy mask allowing to be positioned on an oxygen therapy mask allowing to capture fluid present between the vacuum shield and the therapy oxygen mask.

### CROSS REFERENCE TO RELATED APPLICATION

The present application claims priority to Canadian Patent Application No. 3,080,573 entitled “Vacuum Shield” filed on May 6, 2020, the contents of which are incorporated herein by reference in their entirety.

### FIELD OF THE INVENTION

The present invention pertains to the field of shields and more specifically to a vacuum shield for use with an oxygen therapy mask.

### BACKGROUND OF THE INVENTION

There is a need for increased control of particulates present around a patient in a hospital in order to reduce the chances of a spreading a disease to another patient or person. The use of an oxygen therapy mask is common in hospitals and there is a need for a vacuum shield which can remove particulates present in front of an oxygen therapy mask reducing the chances of harmful diseases transferring to other patients or persons.

### SUMMARY OF THE INVENTION

The present invention provides a vacuum shield for use on an oxygen therapy mask comprising a semi-rigid shield having outer wall regions for covering the oxygen therapy mask and exhaust ports of the oxygen therapy mask. The vacuum shield also has flow ridges positioned on the semi-rigid shield to channel a fluid flow to the outer wall regions of the semi-rigid shield and a vacuum port positioned at a central bottom portion of the semi-rigid shield to draw particulates away from the outer wall regions wherein the vacuum shield can be positioned on an oxygen therapy mask.

### BRIEF DESCRIPTION OF THE DRAWINGS

The embodiments of the present invention will now be described by reference to the following figures, in which identical reference numerals in different figures indicate identical elements and in which:

FIG. 1 is a side view of a common oxygen therapy mask used in hospitals;

FIG. 2 is a perspective view of vacuum shield for use on an oxygen therapy mask according to one embodiment of the present invention;

FIG. 2a is a front view of vacuum shield for use on an oxygen therapy mask according to one embodiment of the present invention;

FIG. 3 is a back view of a vacuum shield with ridges according to one embodiment of the present invention;

FIG. 4 is a side of a vacuum shield positioned on an oxygen therapy mask according to one embodiment of the present invention;

FIG. 5 is a perspective view of a vacuum shield positioned on an oxygen therapy mask according to one embodiment of the present invention;

FIG. 6 is an underside view of a vacuum shield positioned on an oxygen therapy mask with a gap between the vacuum shield and oxygen therapy mask according to one embodiment of the present invention;

The Figures are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art to variously employ the present invention.

### DETAILED DESCRIPTION OF THE INVENTION

The following embodiments are merely illustrative and are not intended to be limiting. It will be appreciated that various modifications and/or alterations to the embodiments described herein may be made without departing from the disclosure and any modifications and/or alterations are within the scope of the contemplated disclosure.

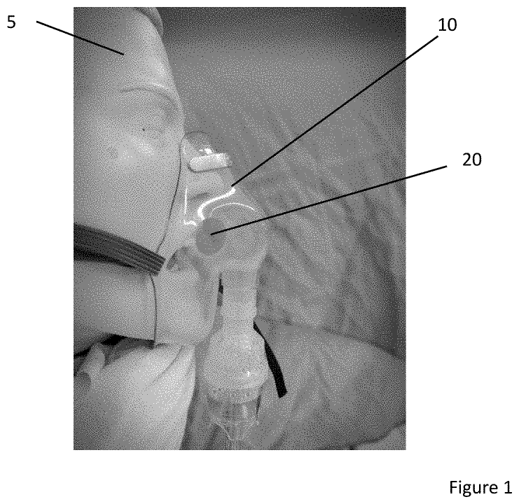

With reference to FIG. 1 a standard oxygen therapy mask 10 is shown installed on a mannequin 5. Such oxygen therapy mask 10 is commonly used in hospitals. The oxygen therapy mask 10 has exhaust ports 20 allowing a person to inhale and exhale while the oxygen therapy mask 10 is positioned on a person. A worker skilled in the relevant art would be familiar with commonly used oxygen therapy mask 10 shown in FIG. 1.

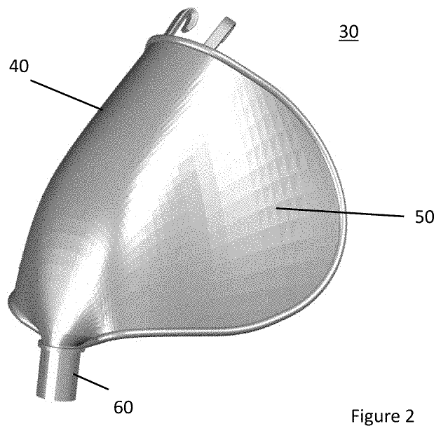

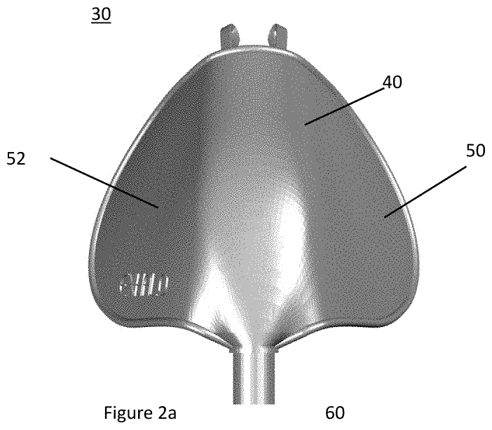

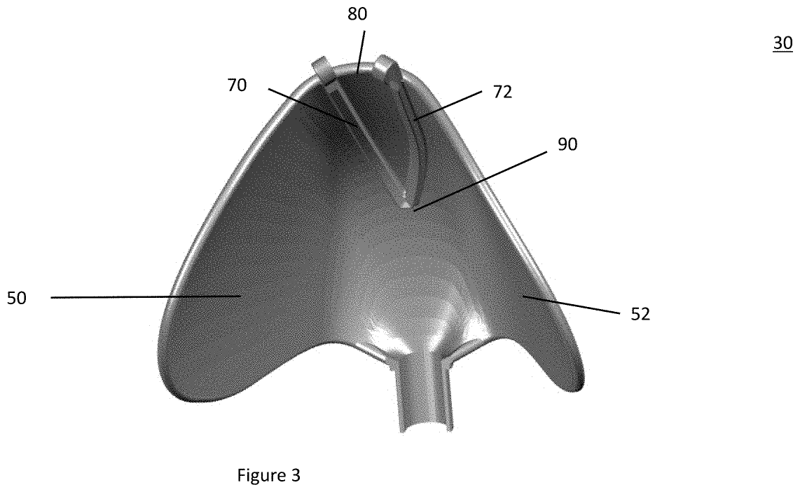

With reference to FIGS. 2 and 3 and according to one embodiment of the present invention, a vacuum shield 30 is shown. Vacuum shield 30 has a semi-rigid shield 40 with outer wall regions 50 and 52. A vacuum port 60 is positioned at a central bottom portion of the semi-rigid shield 40. The vacuum port 60 allows for a conduit to be secured to vacuum port 60 in order to draw air out of the vacuum shield 30. The semi-rigid shield 40 can be composed of polymer or pvc type material. A worker skilled in the relevant art would be familiar with the required composition of the semi-rigid shield in order to provide it with the required rigidity.

With reference to FIG. 3 and according to one embodiment of the present invention, vacuum shield 30 has flow ridges 70 and 72 positioned on interior wall 80. Ridges 70 and 72 provide flow guidance to fluids present in between the vacuum shield 30 and an oxygen therapy mask (not shown). Ridges 70 and 72 channel the flow of any fluids (fluid flow) along the outer wall regions 50 and 52 (within the interior portion of semi-rigid shield 40) which facilitates the drawing of a fluid by vacuum conduit 60. Ridges 70 and 72 are shown in a v-shape position which is not required as ridges 70 and 72 could be parallel to one another. Ridges 70 and 72 extend to a convex tip 80 of semi-rigid shield 40 and ridges are positioned away from the exhaust ports of the oxygen therapy mask (not shown). Ridges 70 and 72 could consist of a single ridge as well rather than two ridges. Two ridges or a single ridge could extend the entire length of the semi-rigid shield 40 and can extend beyond convex tip 80.

A fluid under the present invention is defined as any gas, liquid, bacteria or virus which can be present in the air.

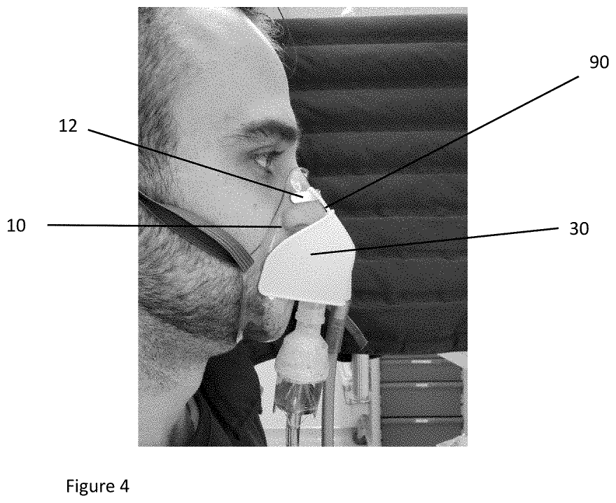

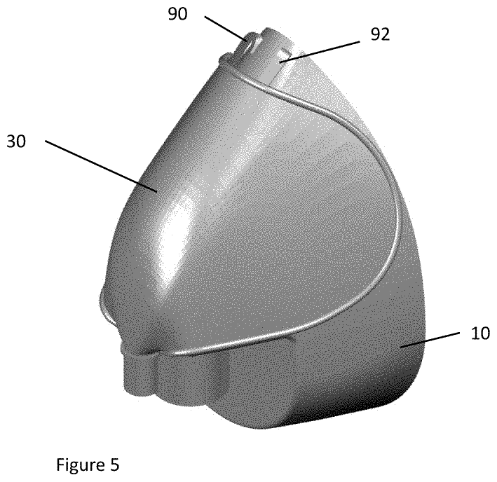

With reference to FIGS. 4 and 5 and according to one embodiment of the present invention, vacuum shield 30 is installed on an oxygen therapy mask 10. Vacuum shield 30 is positioned on the oxygen therapy mask 10 through hook 90 which is attached to metal strip 12 of oxygen therapy mask 10. Vacuum shield 30 can be positioned on oxygen therapy mask 10 in a number of different manners such as through a hook 90 as shown or hooks, a strap, Velcro between the oxygen therapy mask and vacuum shield or any other method which allows the vacuum shield to be positioned on the oxygen therapy mask without falling off the mask.

With further reference to FIG. 5 and according to one embodiment of the present invention, vacuum shield 30 is positioned on an oxygen therapy mask and is not connected to any tube which is part of the oxygen therapy mask 10 or face mask.

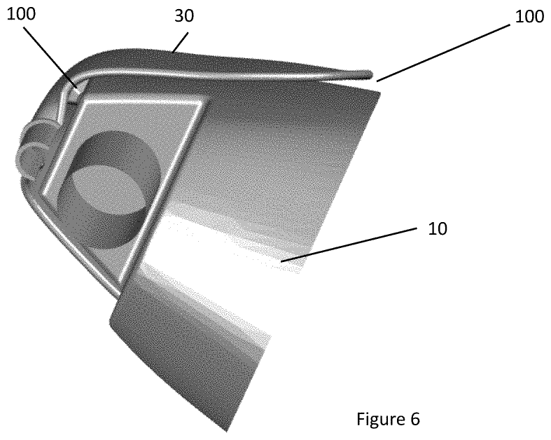

With reference to FIG. 6 and according to one embodiment of the present invention, vacuum shield 30 is positioned on an oxygen therapy mask 10 wherein the vacuum shield 30 has a gap 100 in between the vacuum shield and the oxygen therapy mask 10. Gap 100 is important in order to not seal exhaust ports (not shown) present on oxygen therapy mask 10. The gap 100 can be created through ridges 70 and 72 as shown in FIGS. 2 and 3 or any other ridge enabling a gap to be present between vacuum shield 30 and oxygen therapy mask 10.

A person understanding this invention may now conceive of alternative structures and embodiments or variations of the above all of which are intended to fall within the scope of the invention as defined in the claims that follow.

1. A vacuum shield for use on an oxygen therapy mask comprising:
a semi-rigid shield having outer wall regions for covering the oxygen therapy mask and exhaust ports of the oxygen therapy mask;
one or more flow ridges positioned on the semi-rigid shield to channel a fluid flow to the outer wall regions of the semi-rigid shield;
a vacuum port positioned at a central bottom portion of the semi-rigid shield to draw particulates away from the outer wall regions; and
wherein the vacuum shield can be positioned on an oxygen therapy mask.

2. The vacuum shield according to claim 1 wherein the vacuum shield has two or more ridges.

3. The vacuum shield according to claim 1 wherein the flow ridges extend to a convex tip of the vacuum shield.

4. The vacuum shield according to claim 1 further comprising hooks to secure the vacuum shield to an oxygen therapy mask.

5. The vacuum shield according to claim 1 wherein the one or more ridges create a gap between the vacuum shield and oxygen therapy mask.
